(19) United States
(12) Patent Application Publication
(10) Pub. No.: US 2020/0315505 A1
(43) Pub. Date: Oct. 8, 2020

(54) APPARATUS AND METHOD FOR ESTIMATING ANALYTE CONCENTRATION

(71) Applicant: SAMSUNG ELECTRONICS CO., LTD., Suwon-si (KR)

(72) Inventors: Sang Kyu KIM, Yongin-si (KR); Jun Ho LEE, Incheon (KR)

(73) Assignee: SAMSUNG ELECTRONICS CO., LTD., Suwon-si (KR)

(21) Appl. No.: 16/738,649

(22) Filed: Jan. 9, 2020

(30) Foreign Application Priority Data
Apr. 2, 2019 (KR) 10-2019-0038562

(51) Int. Cl. A61B 5/145; A61B 5/1455

(52) U.S. Cl. CPC A61B 5/14532; A61B 5/1455; A61B 5/14546

(57) ABSTRACT

An apparatus for estimating a concentration of an analyte may include a processor configured to measure a first scattering coefficient of an object at a first wavelength and a second scattering coefficient of the object at a second wavelength, obtain a third scattering coefficient of the object at the third wavelength based on the first scattering coefficient and the second scattering coefficient, measure reflectance of the object at a third wavelength, and estimate the concentration of the analyte based on the obtained third scattering coefficient and the measured reflectance.

### CROSS-REFERENCE TO RELATED APPLICATION(S)

This application is based on and claims priority under 35 U.S.C. § 119 to Korean Patent Application No. 10-2019-0038562, filed on Apr. 2, 2019, in the Korean Intellectual Property Office, the disclosure of which is incorporated by reference herein in its entirety.

### BACKGROUND

### 1. Field

The disclosure relates to an apparatus and method for estimating the concentration of an in vivo analyte.

### 2. Description of Related Art

Diabetes is a chronic disease that causes various complications and can be difficult to cure, such that people with diabetes are advised to check their blood glucose regularly to prevent complications. In particular, when insulin is administered to control blood glucose levels, the blood glucose levels should be closely monitored to avoid hypoglycemia and control insulin dosage. An invasive method of finger pricking is generally used to measure blood glucose levels. However, although the invasive method may provide high reliability in measurement, it may cause pain and inconvenience as well as an increased risk of infection due to the use of injection. Recently, research has been conducted on methods of non-invasively measuring blood glucose by using a spectrometer without blood sampling.

Furthermore, in the case of turbid media such as skin, it may be difficult to estimate an analyte concentration due to path length distribution caused by scattering characteristics. Accordingly, for turbid media, the analyte concentration is estimated by separating an absorption coefficient and a scattering coefficient, and using only the separated absorption coefficient. However, the method may be applied in a visible ray (VIS) region and a short near infrared ray (SNIR) region, to which diffusion approximation is applied, but may not be applied in a near-infrared ray (NIR) region, in which the magnitude of the absorption coefficient is similar to the magnitude of the scattering coefficient, thereby not satisfying the condition of diffusion approximation.

### SUMMARY

Provided are an apparatus and method for estimating the concentration of an in vivo analyte.

Additional aspects will be set forth in part in the description which follows and, in part, will be apparent from the description, or may be learned by practice of the presented embodiments.

According to an aspect of the disclosure, an apparatus for estimating a concentration of an analyte may include a processor configured to drive a first optical sensor that is configured to emit at least two light beams, including light of a first wavelength and light of a second wavelength, toward an object and receive light reflected by the object, measure a first scattering coefficient of the object at the first wavelength and a second scattering coefficient of the object at the second wavelength, obtain a third scattering coefficient of the object at a third wavelength based on the first scattering coefficient and the second scattering coefficient, drive a second optical sensor configured to emit at least one light beam, including light of the third wavelength, toward the object and receive light reflected by the object, measure reflectance of the object at the third wavelength, and estimate the concentration of the analyte based on the obtained third scattering coefficient and the measured reflectance.

The first wavelength may be in a visible ray (VS) region, and the second wavelength may be in a short near infrared ray (SNIR) region.

The third wavelength may be a wavelength in a near-infrared ray (NR) region, and may be longer than a wavelength in a short near infrared ray (SNIR) region.

The first optical sensor may implement at least one of spatially resolved spectroscopy, time resolved spectroscopy, and frequency modulation spectroscopy, and the processor may measure the first scattering coefficient and the second scattering coefficient by using at least one of spatially resolved spectroscopy, time resolved spectroscopy, and frequency modulation spectroscopy.

The processor may obtain the third scattering coefficient from the first scattering coefficient and the second scattering coefficient by using monotonicity properties of the first scattering coefficient and the second scattering coefficient.

The processor may derive a monotonic function, representing the monotonicity properties of the first scattering coefficient and the second scattering coefficient, based on the first scattering coefficient and the second scattering coefficient, and obtain the third scattering coefficient by using the derived monotonic function.

The processor may obtain an absorption coefficient of the object at the third wavelength based on the obtained third scattering coefficient and the reflectance, and estimate the concentration of the analyte by analyzing the obtained absorption coefficient.

The processor may obtain an albedo of the object at the third wavelength based on the reflectance, and obtain the absorption coefficient based on the obtained albedo and the third scattering coefficient.

The analyte may be at least one of glucose, triglyceride, urea, uric acid, lactate, protein, cholesterol, and ethanol.

A method of estimating a concentration of an analyte may include measuring a first scattering coefficient of an object at a first wavelength, measuring a second scattering coefficient of the object at a second wavelength, obtaining a third scattering coefficient of the object at a third wavelength based on the measured first scattering coefficient and the measured second scattering coefficient, measuring reflectance of the object at the third wavelength and estimating the concentration of the analyte based on the obtained third scattering coefficient and the measured reflectance.

The first wavelength may be in a visible ray (VIS) region, and the second wavelength may be in a short near infrared ray (SNIR) region.

The third wavelength may be a wavelength in a near-infrared ray (NIR) region, and may be longer than a wavelength in a short near infrared ray (SNIR) region.

The measuring of the first scattering coefficient and the measuring of the second scattering coefficient may respectively include measuring the first scattering coefficient and the second scattering coefficient by using at least one of spatially resolved spectroscopy, time resolved spectroscopy, and frequency modulation spectroscopy.

The obtaining of the third scattering coefficient may include obtaining the third scattering coefficient from the first scattering coefficient and the second scattering coefficient by using monotonicity properties of the first scattering coefficient and the second scattering coefficient.

The obtaining of the third scattering coefficient may include deriving a monotonic function, representing the monotonicity properties of the first scattering coefficient and the second scattering coefficient, based on the first scattering coefficient and the second scattering coefficient, and obtaining the third scattering coefficient by using the derived monotonic function.

The estimating of the concentration of the analyte may include obtaining an absorption coefficient of the object at the third wavelength based on the obtained third scattering coefficient and the measured reflectance, and estimating the concentration of the analyte by analyzing the obtained absorption coefficient.

The obtaining of the absorption coefficient may include obtaining an albedo of the object at the third wavelength based on the reflectance, and obtaining the absorption coefficient based on the obtained albedo and the third scattering coefficient.

The analyte may be at least one of glucose, triglyceride, urea, uric acid, lactate, protein, cholesterol, and ethanol.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram illustrating an example of an apparatus for estimating a concentration of an analyte according to an embodiment;

FIG. 2 is a block diagram illustrating an example of a structure of a first optical sensor according to an embodiment;

FIG. 3 is a block diagram illustrating another example of a structure of a first optical sensor according to an embodiment;

FIG. 4 is a block diagram illustrating yet another example of a structure of a first optical sensor according to an embodiment;

FIG. 5 is a block diagram illustrating still another example of a structure of a first optical sensor according to an embodiment:

FIG. 6 is a block diagram illustrating an example of a structure of a second optical sensor according to an embodiment:

FIG. 7 is a diagram explaining monotonicity properties of a scattering coefficient according to an embodiment;

FIG. 8 is a block diagram illustrating another example of an apparatus for estimating a concentration of an analyte according to an embodiment;

FIG. 9 is a flowchart illustrating an example of a method of estimating an analyte concentration according to an embodiment; and

FIG. 10 is a diagram illustrating an example of a wrist-type wearable device according to an embodiment.

Throughout the drawings and the detailed description, unless otherwise described, the same reference numerals may refer to the same elements, features, and structures. The relative size and depiction of the elements, features, and structures may be exaggerated for clarity, illustration, and convenience.

### DETAILED DESCRIPTION

Hereinafter, embodiments of the present disclosure will be described in detail with reference to the accompanying drawings. It should be noted that wherever possible, the same reference numbers may refer to the same parts even in different drawings. In the following description, a detailed description of known functions and configurations incorporated herein may be omitted so as to not obscure the subject matter of the present disclosure.

Process steps described herein may be performed differently from a specified order, unless a specified order is clearly stated in the context of the disclosure. That is, each step may be performed in a specified order, at substantially the same time, or in a reverse order.

Further, the terms used throughout this specification may be defined in consideration of the functions according to exemplary embodiments, and can be varied according to a purpose of a user or manager, or precedent and so on. Therefore, definitions of the terms should be made on the basis of the overall context of the disclosure.

It should be understood that, although terms such as “first,” “second,” etc., may be used herein to describe various elements, these elements should not be limited by these terms. These terms may be used to distinguish one element from another. Any references to the singular forms of terms may include the plural forms of the terms unless expressly stated otherwise. In the present disclosure, it should be understood that terms such as “including,” “having.” etc., may indicate the existence of the features, numbers, steps, actions, components, parts, or combinations thereof disclosed in the disclosure, and might not preclude the possibility that one or more other features, numbers, steps, actions, components, parts, or combinations thereof may exist or may be added.

Further, components described in the disclosure may be discriminated according to functions mainly performed by the components. That is, two or more components may be integrated into a single component. Furthermore, a single component may be separated into two or more components. Moreover, each component may additionally perform some or all of a function executed by another component in addition to the main function thereof. Some or all of the main function of each component may be carried out by another component. Each component may be implemented in hardware, software, or a combination of both.

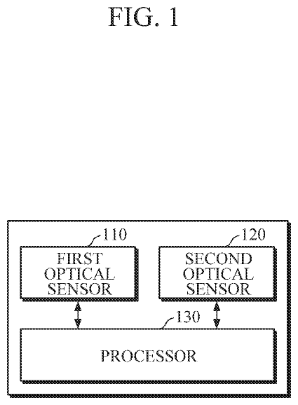

FIG. 1 is a block diagram illustrating an example of an apparatus for estimating a concentration of an analyte according to an embodiment. The apparatus 100 for estimating an analyte concentration of FIG. 1 may be embedded in an electronic device or may be enclosed in a housing to be provided as a separate device. In this case, examples of the electronic device may include a cellular phone, a smartphone, a tablet personal computer (PC), a laptop computer, a personal digital assistant (PDA), a portable multimedia player (PMP), a navigation device, an MP3 player, a digital camera, a wearable device, and the like; and examples of the wearable device may include a wristwatch-type wearable device, a wristband-type wearable device, a ring-type wearable device, a waist belt-type wearable device, a necklace-type wearable device, an ankle band-type wearable device, a thigh band-type wearable device, a forearm band-type wearable device, and the like. However, the electronic device is not limited to the above examples, and the wearable device is neither limited thereto.

Referring to FIG. 1, the apparatus 100 for estimating an analyte concentration includes a first optical sensor 110, a second optical sensor 120, and a processor 130.

The first optical sensor 110 may emit light of a first wavelength and a second wavelength towards an object, and may receive light reflected by or returning from the object. The first wavelength and the second wavelength, which may be different from each other, may be wavelengths in a visible ray (VIS) region and a short near infrared ray (SNIR) region, to which diffusion approximation is applied. For example, the first wavelength and the second wavelength may be wavelengths in a range of 400 nm to 1000 nm.

In an embodiment, the first optical sensor 110 may have a structure in which at least one of spatially resolved spectroscopy, time resolved spectroscopy, and frequency modulation spectroscopy may be used.

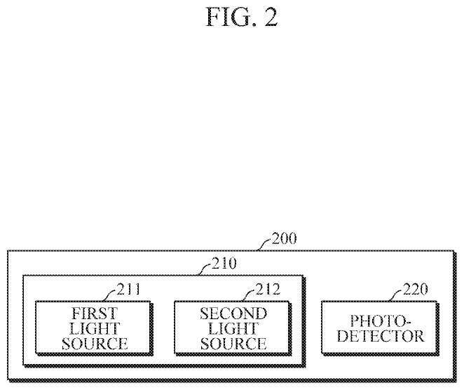

Hereinafter, examples of the first optical sensor 110 will be described in detail with reference to FIGS. 2 to 5.

FIG. 2 is a block diagram illustrating an example of a structure of a first optical sensor according to an embodiment. The first optical sensor 200 of FIG. 2 is an example of the optical sensor 110 of FIG. 1, and has a structure in which time resolved spectroscopy or frequency modulation spectroscopy may be used.

Referring to FIG. 2, the first optical sensor 200 includes a light source assembly 210, including a plurality of light sources 211 and 212, and a photodetector 220. FIG. 2 illustrates an example in which the light source assembly 210 includes two light sources 211 and 212, but this is merely for convenience of explanation and is not intended to be limiting, and there is no specific limitation on the number of the light sources.

The first light source 211 may emit light of the first wavelength toward the object, and the second light source 212 may emit light of the second wavelength toward the object. In this case, the first wavelength and the second wavelength, which may be different from each other, may be wavelengths in a visible ray (VIS) region and a short near infrared ray (SNIR) region, to which diffusion approximation is applied.

In an embodiment, the first light source 211 and the second light source 212 may include a light emitting diode (LED), an organic light emitting diode (OLED), a quantum dot light-emitting diode (QLED), a laser diode, a phosphor, and the like, but are not limited thereto.

The photodetector 220 may receive light of the first wavelength which is emitted by the first light source 211 and reflected by the object, and may receive light of the second wavelength which is emitted by the second light source 212 and reflected by the object. In an embodiment, the photodetector 220 may include a photo diode, a photo transistor, a charge-coupled device (DDC) a complementary metal-oxide semiconductor (CMOS), and the like, but is not limited thereto.

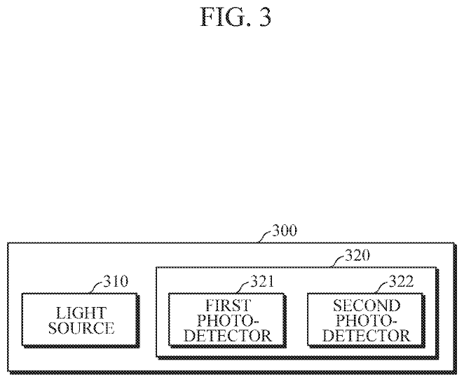

FIG. 3 is a block diagram illustrating another example of a structure of a first optical sensor according to an embodiment. The first optical sensor 300 of FIG. 3 is an example of the optical sensor 110 of FIG. 1, and has a structure in which time resolved spectroscopy or frequency modulation spectroscopy may be used.

Referring to FIG. 3, the first optical sensor 300 includes a light source 310 and a photodetector assembly 320. The photodetector assembly 320 includes a first photodetector 321 and a second photodetector 322. Although FIG. 3 illustrates an example in which the photodetector assembly 320 includes two photodetectors 321 and 322, this is merely for convenience of explanation and not intended to be limiting, and there is no specific limitation on the number of the photodetectors.

The light source 310 may emit light of a predetermined wavelength toward the object. For example, the light source 310, which is a single light source, may emit light in a wide wavelength range, including a visible ray (VIS) region and/or a short near infrared ray (SNIR) region, to which diffusion approximation is applied.

The photodetector assembly 320 may receive light in a predetermined wavelength range which is reflected by the object. In this case, the photodetector assembly 320 may have different response characteristics.

For example, the first photodetector 321 and the second photodetector 322 may have different measurement ranges so as to respond to light of different wavelengths, among light beams in a predetermined wavelength range which are reflected by the object. Alternatively, in order to respond to light of different wavelengths, a filter may be provided on a front surface of any one of the first photodetector 321 and the second photodetector 322, or different filters may be provided on front surfaces of the two photodetectors 321 and 322.

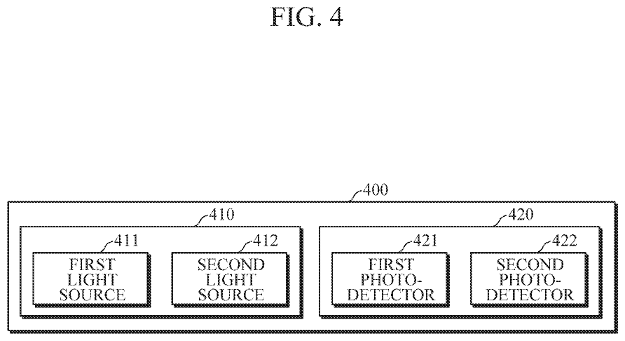

FIG. 4 is a block diagram illustrating yet another example of a structure of a first optical sensor according to an embodiment. The first optical sensor 400 of FIG. 4 is an example of the optical sensor 110 of FIG. 1, and has a structure in which spatially resolved spectroscopy may be used.

Referring to FIG. 4, the first optical sensor 400 includes a light source assembly 410 including a plurality of light sources 411 and 412, and a photodetector assembly 320 including a plurality of photodetectors 421 and 422. Although FIG. 4 illustrates an example in which the light source assembly 410 includes two light sources 411 and 412, and the photodetector assembly 420 includes two photodetectors 421 and 422, this is merely for convenience of explanation and not intended to be limiting, and there is no specific limitation on the number of the light sources and the photodetectors.

The first light source 411 may emit light of a first wavelength towards an object, and the second light source 412 may emit light of a second wavelength towards an object. In this case, the first wavelength and the second wavelength, which may be different from each other, may be wavelengths in a visible ray (VIS) region and a short near infrared ray (SNIR) region, to which diffusion approximation is applied.

The first photodetector 421 and the second photodetector 422 may receive light of the first wavelength, which is emitted by the first light source 411 and reflected by the object, and light of the second wavelength, which is emitted by the second light source 412 and reflected by the object, respectively. The first photodetector 421 and the second photodetector 422 may be positioned at different distances from each of the light sources 411 and 412.

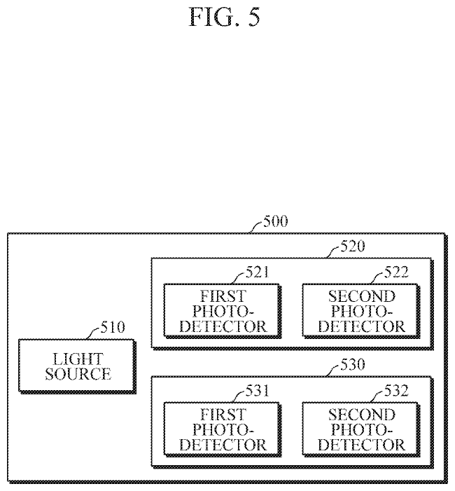

FIG. 5 is a block diagram illustrating still another example of a structure of a first optical sensor according to an embodiment. The first optical sensor 500 of FIG. 5 is an example of the optical sensor 110 of FIG. 1, and has a structure in which spatially resolved spectroscopy may be used.

Referring to FIG. 5, the first optical sensor 500 includes a light source 510, a first photodetector assembly 520 and a second photodetector assembly 530. The first photodetector assembly 520 includes a first photodetector 521 and a second photodetector 522, and the second photodetector assembly 530 includes a first photodetector 531 and a second photodetector 530. Although FIG. 5 illustrates an example in which the optical sensor 500 includes two photodetector assemblies 520 and 530, each of which includes two photodetectors, this is merely for convenience of explanation and not intended to be limiting, and there is no specific limitation on the number of the photodetector assemblies and the photodetectors included in each of the photodetector assemblies.

The light source 510 may emit light of a predetermined wavelength toward the object. For example, the light source 510, which is a single light source, may emit light in a wide wavelength range including a visible ray (VIS) region and/or a short near infrared ray (SNIR) region, to which diffusion approximation is applied.

The first photodetector assembly 520 and the second photodetector assembly 530 may receive light in a predetermined wavelength range which is reflected by the object. In this case, the photodetector assemblies 520 and 530 may be formed to have different response characteristics.

For example, the first photodetector assembly 520 and the second photodetector assembly 530 may have different measurement ranges so as to respond to light of different wavelengths, among light beams in a predetermined wavelength range which are reflected by the object. Alternatively, in order to respond to light of different wavelengths, a filter may be provided on a front surface of any one of the first photodetector assembly 520 and the second photodetector assembly 530, or different filters may be provided on front surfaces of the two photodetector assemblies 520 and 530. In this case, photodetectors included in the same photodetector assembly may be provided with the same filter.

The first photodetector 521 and the second photodetector 522 of the first photodetector assembly 520 may be positioned at different distances from the light source 510, and the first photodetector 521 and the second photodetector 522 of the second photodetector assembly 530 may be positioned at different distances from the light source 510.

Referring to FIG. 1, the second optical sensor 120 may emit light of a third wavelength toward an object, and may receive light reflected by the object. Here, the third wavelength may be a wavelength in the near-infrared ray (NIR) region, which is longer than a wavelength in the SNIR region to which diffusion approximation is not applied. For example, the third wavelength may be a wavelength in a range of 1500 nm to 1800 nm.

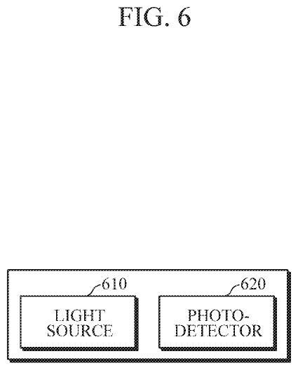

Hereinafter, an example of the second optical sensor 120 will be described in detail with reference to FIG. 6.

FIG. 6 is a block diagram illustrating an example of a structure of a second optical sensor according to an embodiment. The second optical sensor 600 of FIG. 6 may be an example of the second optical sensor 120 of FIG. 1.

Referring to FIG. 6, the second optical sensor 600 includes a light source 610 and a photodetector 620. Although FIG. 6 illustrates an example in which the second optical sensor 600 includes one light source 610 and one photodetector 620, this is merely for convenience of explanation and not intended to be limiting, and there is no specific limitation on the number of the light source 610 and the photodetector 620.

The light source 610 may emit light of a third wavelength toward an object. The light source 610 may be a single light-emitting body, or may be formed of an array of a plurality of light-emitting bodies. In the case where the light source 610 is formed of a plurality of light-emitting bodies, the plurality of light-emitting bodies may emit light of different wavelengths or may emit light of the same wavelength. In an embodiment, examples of the light source 610 may include a light emitting diode (LED), an organic light emitting diode (OLED), a quantum dot light-emitting diode (QLED), a laser diode, a phosphor, and the like, but the light source 610 is not limited thereto.

The photodetector 620 may receive light reflected by the object. The photodetector 620 may be a single device, or may be formed of an array of a plurality of devices. In an embodiment, examples of the photodetector 620 may include a photo diode, a photo transistor (PTr), a charge-coupled device (CCD), a Complementary Metal-Oxide Semiconductor (CMOS), and the like, but the photodetector 620 is not limited thereto.

As illustrated in FIGS. 1 to 6, the first optical sensor 110 and the second optical sensor 120 may be implemented as separate devices, but are not limited thereto. That is, the first optical sensor 110 and the second optical sensor 120 may share some of the light sources or some of the photodetectors.

Referring back to FIG. 1, the processor 130 may control the overall operation of the apparatus 100 for estimating an analyte concentration.

In response to an occurrence of a predetermined event, the processor 130 may drive the first optical sensor 110 to measure a first scattering coefficient of an object at a first wavelength and a second scattering coefficient of the object at a second wavelength. In this case, the processor 130 may obtain the first scattering coefficient and the second scattering coefficient by using at least one of spatially resolved spectroscopy, time resolved spectroscopy, and frequency modulation spectroscopy.

The processor 130 may obtain a third scattering coefficient of the object at a third wavelength based on the first scattering coefficient and the second scattering coefficient. In this case, the third wavelength may be a wavelength in the near-infrared ray (NIR) region, which is longer than a wavelength in the SNIR region to which diffusion approximation is not applied. For example, the third wavelength may be a wavelength in a range of 1500 nm to 1800 nm.

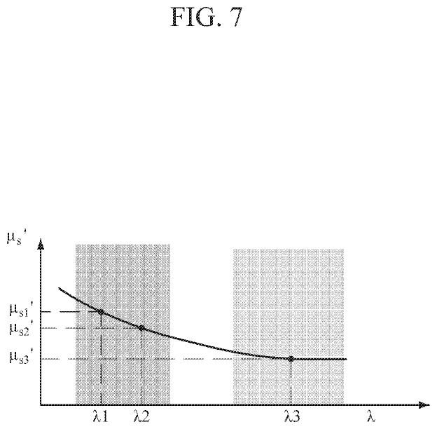

In an embodiment, the processor 130 may obtain the third scattering coefficient from the first scattering coefficient and the second scattering coefficient by using monotonicity properties of the scattering coefficient. As illustrated in FIG. 7, a reduced scattering coefficient may have monotonicity properties, in which the scattering coefficient decreases as wavelength increases. Such monotonicity properties of the scattering coefficient may be expressed in the form of a monotonic function by using the following Equation 1. However, Equation 1 is merely an example. That is, the monotonic function, representative of monotonicity properties of the scattering coefficient, may be represented in various forms.

μ′ₛ(λ)=Aλ^{-B} [Equation 1]

Referring to Equation 1, λ denotes the wavelength; μ′ₛ(λ) denotes the scattering coefficient at the wavelength of λ; and A and B denote coefficients related to scatter density and Mie scatter size respectively.

In an embodiment, the processor 130 may derive a monotonic function, representing monotonicity properties of the scattering coefficient, based on the first scattering coefficient and the second scattering coefficient, and may obtain the third scattering coefficient of the object at the third wavelength by using the derived monotonic function. For example, the processor 130 may obtain Equation 1 by determining A and B of Equation 1 by using the first scattering coefficient and the second scattering coefficient, and may obtain the third scattering coefficient of the object at the third wavelength by using the obtained Equation 1.

The processor 130 may drive the second optical sensor 120 to measure reflectance of the object at the third wavelength. For example, the processor 130 may measure the reflectance of the object at the third wavelength by using an amount of incident light, emitted by the second optical sensor 120 toward the object, and an amount of reflected light reflected by the object.

The processor 130 may obtain an absorption coefficient at the third wavelength based on the reflectance and the third scattering coefficient at the third wavelength. In an embodiment, the processor 130 may obtain an albedo at the third wavelength by using the following Equation 2 based on the reflectance at the third wavelength, and may obtain the absorption coefficient at the third wavelength by using the following Equation 3 based on the obtained albedo and the third scattering coefficient at the third wavelength.

$$R = \frac{{a}^{′}}{1 + 2 k ( 1 - {a}^{′} ) + ( 1 + \frac{2 k}{3} ) \sqrt{3 ( 1 - {a}^{′} )}} \\ k = \frac{1 + {r}_{d}}{1 - {r}_{d}} \\ {r}_{d} = - 1.440 {n}_{rel}^{- 2} + 0.710 {n}_{rel}^{- 1} + 0.668 + 0.0636 {n}_{rel} \qquad [ Equation 2 ]$$

Referring to Equation 2, R denotes the reflectance, a′ denotes the albedo, and nᵣₑₗ denotes a refractive index.

$${μ}_{a} = ( \frac{1}{{a}^{′}} - 1 ) {μ}_{s}^{′} \qquad [ Equation 3 ]$$

Referring to Equation 3, μₐ denotes the absorption coefficient, and μ′ₛ denotes the scattering coefficient.

The processor 130 may estimate the concentration of an analyte by analyzing the absorption coefficient at the third wavelength. In this case, the analyte may include glucose, triglyceride, urea, uric acid, lactate, protein, cholesterol, ethanol, and the like, but is not limited thereto.

The absorption coefficient may be represented by the following Equation 4.

μₐ=ε₁C₁+ε₂C₂+ε₃C₃+ [Equation 4]

Referring to Equation 4, ε denotes the absorption coefficient of each substance, and C denotes the concentration of each substrate.

In an embodiment, the processor 130 may estimate the concentration of an analyte by using the absorption coefficient at the third wavelength and Equation 4. In this case, the processor 130 may use various analysis techniques such as regression analysis, Classical Least Squares (CLS), Net Analyte Signal (NAS) algorithm, and the like.

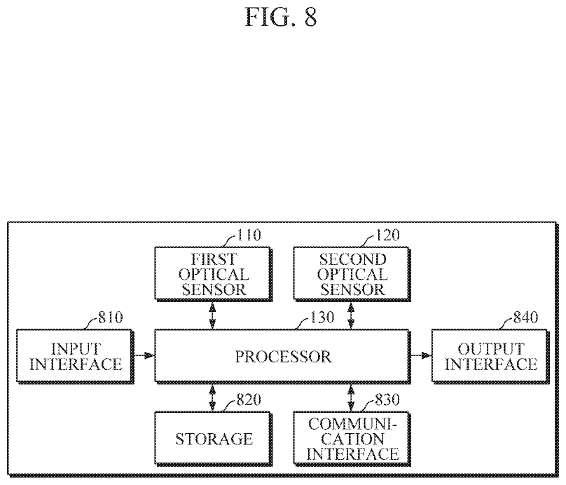

FIG. 8 is a block diagram illustrating another example of an apparatus for estimating a concentration of an analyte. The apparatus 800 for estimating an analyte concentration of FIG. 8 may be embedded in an electronic device or may be enclosed in a housing to be provided as a separate device. In this case, examples of the electronic device may include a cellular phone, a smartphone, a tablet PC, a laptop computer, a personal digital assistant (PDA), a portable multimedia player (PMP), a navigation device, an MP3 player, a digital camera, a wearable device, and the like; and examples of the wearable device may include a wristwatch-type wearable device, a wristband-type wearable device, a ring-type wearable device, a waist belt-type wearable device, a necklace-type wearable device, an ankle band-type wearable device, a thigh band-type wearable device, a forearm band-type wearable device, and the like. However, the electronic device is not limited to the above examples, and the wearable device is neither limited thereto.

Referring to FIG. 8, the apparatus 100 for estimating an analyte concentration includes a first optical sensor 110, a second optical sensor 120, a processor 130, an input interface 810, a storage 820, a communication interface 830, and an output interface 840. Here, the first optical sensor 110, the second optical sensor 120, and the processor 130 may be substantially the same as the first optical sensor 110, the second optical sensor 120, and the processor 130 described above with reference to FIGS. 1 to 7, such that detailed description thereof may be omitted.

The input interface 810 may receive input of various operation signals from a user based on a user input. In an embodiment, the input interface 810 may include a keypad, a dome switch, a touch pad (e.g., a static pressure touch pad, a capacitive touch pad, etc.), a jog wheel, a jog switch, a hardware (H/W) button, and the like. Particularly, the touch pad, which forms a layer structure with a display, may be referred to as a touch screen.

The storage 820 may store programs or instructions for operation of the apparatus 800 for estimating an analyte concentration, and may store data input to the apparatus 800 for estimating an analyte concentration, data processed and output by the apparatus 800 for estimating an analyte concentration, and the like. The storage 820 may include at least one storage medium of a flash memory type memory, a hard disk type memory, a multimedia card micro type memory, a card type memory (e.g., a secure digital (SD) memory, an extreme digital (XD) memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like. Further, the apparatus 800 for estimating an analyte concentration may communicate with an external storage medium, such as web storage and the like, which performs a storage function of the storage 820 on the Internet.

The communication interface 830 may perform communication with an external device. For example, the communication interface 830 may transmit, to the external device, data input to and stored in the apparatus 800 for estimating an analyte concentration, data processed by the apparatus 800 for estimating an analyte concentration, and the like; or may receive, from the external device, various data for estimating an analyte concentration.

In this case, the external device may be medical equipment using the data input to and stored in the apparatus 800 for estimating an analyte concentration, the data processed by the apparatus 800 for estimating an analyte concentration, and the like, a printer to print out results, or a display to display the results. In addition, the external device may be a digital television (TV), a desktop computer, a cellular phone, a smartphone, a tablet PC, a laptop computer, a personal digital assistant (PDA), a portable multimedia player (PMP), a navigation, an MP3 player, a digital camera, a wearable device, and the like, but the external device is not limited thereto.

The communication interface 830 may communicate with an external device by using Bluetooth communication, Bluetooth Low Energy (BLE) communication, Near Field Communication (NFC), WLAN communication, Zigbee communication, Infrared Data Association (IrDA) communication, wireless fidelity (Wi-Fi) Direct (WFD) communication, Ultra-Wideband (UWB) communication, Ant+ communication, Wi-Fi communication, Radio Frequency Identification (RFID) communication, third generation (3G) communication, fourth generation (4G) communication, fifth generation (5G) communication, and the like. However, this is merely exemplary and not intended to be limiting.

The output interface 840 may output the data input to and stored in the apparatus 800 for estimating an analyte concentration, the data processed by the apparatus 800 for estimating an analyte concentration, and the like. In an embodiment, the output interface 840 may output the data input to and stored in the apparatus 800 for estimating an analyte concentration, the data processed by the apparatus 800 for estimating an analyte concentration, and the like, by using at least one of an acoustic method, a visual method, and a tactile method. To this end, the output interface 860 may include a display, a speaker, a vibrator, and the like.

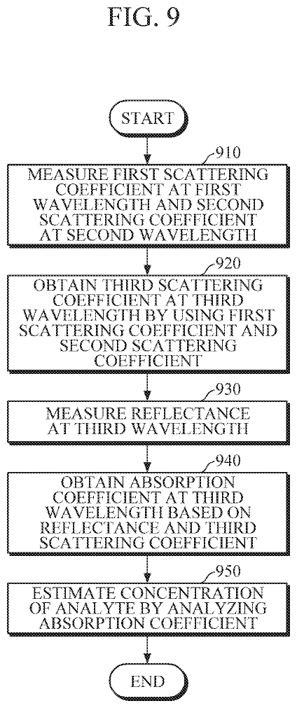

FIG. 9 is a flowchart illustrating an example of a method of estimating an analyte concentration. The method of estimating an analyte concentration of FIG. 9 may be performed by the apparatuses 100 and 800 of estimating an analyte concentration of FIGS. 1 and 8.

Referring to FIG. 9, the apparatus for estimating an analyte concentration may measure a first scattering coefficient of an object at a first wavelength and a second scattering coefficient of the object at a second wavelength in operation 910. For example, the apparatus for estimating an analyte coefficient may obtain the first scattering coefficient and the second scattering coefficient by using at least one of spatially resolved spectroscopy, time resolved spectroscopy, and frequency modulation spectroscopy. In this case, the first wavelength and the second wavelength, which may be different from each other, may be wavelengths in a visible ray (VIS) region and a short near infrared ray (SNIR) region, to which diffusion approximation is applied. For example, the first wavelength and the second wavelength may be wavelengths in a range of 400 nm to 1000 nm.

The apparatus for estimating an analyte concentration may obtain a third scattering coefficient of an object at a third wavelength by using the first scattering coefficient and the second scattering coefficient in operation 920. In this case, the third wavelength may be a wavelength in a near-infrared ray (NIR) region, to which diffusion approximation is not applied. For example, the third wavelength may be a wavelength in a range of 1500 nm to 1800 nm.

In an embodiment, the apparatus for estimating an analyte concentration may obtain the third scattering coefficient from the first scattering coefficient and the second scattering coefficient using monotonicity properties of the scattering coefficient. For example, the apparatus for estimating an analyte concentration may obtain the third scattering coefficient of the object at the third wavelength using Equation 1 shown elsewhere herein above.

The apparatus for estimating an analyte concentration may measure reflectance of the object at the third wavelength in operation 930.

The apparatus for estimating an analyte concentration may obtain an absorption coefficient at the third wavelength based on the reflectance and the third scattering coefficient at the third wavelength in operation 940. In an embodiment, the apparatus for estimating an analyte concentration may obtain an albedo at the third wavelength using Equation 2 shown elsewhere herein above based on the reflectance at the third wavelength, and may obtain the absorption coefficient at the third wavelength using Equation 3 shown elsewhere herein above based on the obtained albedo and the third scattering coefficient at the third wavelength.

The apparatus for estimating an analyte concentration may estimate the concentration of an analyte by analyzing the absorption coefficient at the third wavelength in operation 950. In this case, examples of the analyte may include glucose, triglyceride, urea, uric acid, lactate, protein, cholesterol, ethanol, and the like, but is not limited thereto. In an embodiment, the apparatus for estimating an analyte concentration may estimate the concentration of an analyte by using the absorption coefficient at the third wavelength and Equation 4 shown elsewhere herein above. In this case, the apparatus for estimating an analyte concentration may use various analysis techniques such as regression analysis, Classical Least Squares (CLS), Net Analyte Signal (NAS) algorithm, and the like.

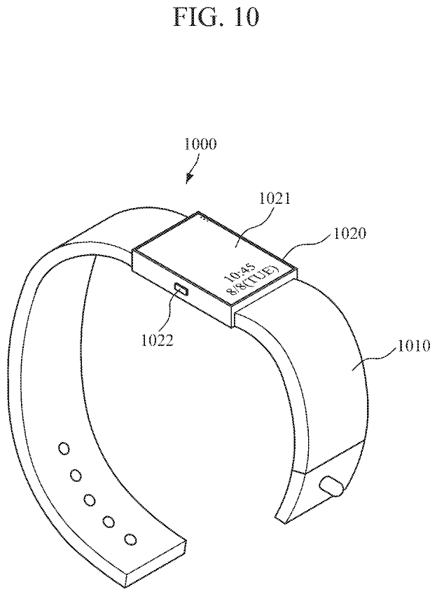

FIG. 10 is a diagram illustrating an example of a wrist-type wearable device.

Referring to FIG. 10, the wrist-type wearable device 1000 includes a strap 1010 and a main body 1020.

The strap 1010 may be connected to both ends of the main body 1020 so as to be fastened in a detachable manner or may be integrally formed therewith as a smart band. The strap 1010 may be made of a flexible material to be wrapped around a user's wrist so that the main body 1020 may be worn on the wrist.

The main body 1020 may include the apparatuses 100 and 800 for estimating an analyte concentration described above. Further, the main body 1020 may include a battery which supplies power to the wrist-type wearable device 1000 and the apparatuses 100 and 800 for estimating an analyte concentration.

A first optical sensor and a second optical sensor may be provided at the bottom of the main body 1020 to be exposed to a user's wrist. Accordingly, when a user wears the wrist-type wearable device 1000, the first optical sensor and the second optical sensor may contact the user's skin. In this case, the first optical sensor and the second optical sensor may emit light toward an object, and may receive light reflected by or scattered from the object.

The wrist-type wearable device 1000 may further include a display 1021 and an input interface 1022 which are provided in the main body 1020. The display 1021 may display data processed by the wrist-type wearable device 1000 and the apparatuses 100 and 800 for estimating an analyte concentration, processing result data thereof, and the like. The input interface 1022 may receive various operation signals from a user based on a user input.

The embodiments of the present disclosure may be realized as a computer-readable code stored on a non-transitory computer-readable medium. The computer-readable medium may be any type of recording device in which data is stored in a computer-readable manner. Examples of the computer-readable medium include a ROM, a RAM, a CD-ROM, a magnetic tape, a floppy disc, an optical data storage, and a carrier wave (e.g., data transmission via the Internet). The computer-readable recording medium may be distributed via a plurality of computer systems connected to a network so that a computer-readable code is written thereto and executed therefrom in a decentralized manner.

The present disclosure has been described herein with regard to various embodiments. However, it should be apparent to those skilled in the art that various changes and modifications can be made without departing from the scope of the present disclosure. Thus, it is clear that the above-described embodiments are illustrative in all aspects and are not intended to limit the present disclosure.

What is claimed is:

1. An apparatus for estimating a concentration of an analyte, the apparatus comprising:
a processor configured to:
drive a first optical sensor that is configured to emit at least two light beams, including light of a first wavelength and light of a second wavelength, toward an object and receive light reflected by the object;
measure a first scattering coefficient of the object at the first wavelength and a second scattering coefficient of the object at the second wavelength;
obtain a third scattering coefficient of the object at a third wavelength based on the first scattering coefficient and the second scattering coefficient;
drive a second optical sensor configured to emit at least one light beam, including light of the third wavelength, toward the object and receive light reflected by the object;
measure reflectance of the object at the third wavelength; and
estimate the concentration of the analyte based on the obtained third scattering coefficient and the measured reflectance.

2. The apparatus of claim 1, wherein the first wavelength is in a visible ray (VIS) region, and the second wavelength is in a short near infrared ray (SNIR) region.

3. The apparatus of claim 1, wherein the third wavelength is a wavelength in a near-infrared ray (NIR) region, which is longer than a wavelength in a short near infrared ray (SNIR) region.

4. The apparatus of claim 1, wherein:
the first optical sensor is configured to implement at least one of spatially resolved spectroscopy, time resolved spectroscopy, and frequency modulation spectroscopy, and
the processor is configured to measure the first scattering coefficient and the second scattering coefficient by using at least one of spatially resolved spectroscopy, time resolved spectroscopy, and frequency modulation spectroscopy.

5. The apparatus of claim 1, wherein the processor is configured to obtain the third scattering coefficient from the first scattering coefficient and the second scattering coefficient by using monotonicity properties of the first scattering coefficient and the second scattering coefficient.

6. The apparatus of claim 5, wherein the processor is configured to derive a monotonic function, representing the monotonicity properties of the first scattering coefficient and the second scattering coefficient, based on the first scattering coefficient and the second scattering coefficient, and obtain the third scattering coefficient by using the derived monotonic function.

7. The apparatus of claim 1, wherein the processor is configured to:
obtain an absorption coefficient of the object at the third wavelength based on the obtained third scattering coefficient and the reflectance; and
estimate the concentration of the analyte by analyzing the obtained absorption coefficient.

8. The apparatus of claim 7, wherein the processor is configured to:
obtain an albedo of the object at the third wavelength based on the reflectance; and
obtain the absorption coefficient based on the obtained albedo and the third scattering coefficient.

9. The apparatus of claim 1, wherein the analyte is at least one of glucose, triglyceride, urea, uric acid, lactate, protein, cholesterol, and ethanol.

10. A method of estimating a concentration of an analyte, the method comprising:
measuring a first scattering coefficient of an object at a first wavelength;
measuring a second scattering coefficient of the object at a second wavelength;
obtaining a third scattering coefficient of the object at a third wavelength based on the measured first scattering coefficient and the measured second scattering coefficient;
measuring reflectance of the object at the third wavelength; and
estimating the concentration of the analyte based on the obtained third scattering coefficient and the measured reflectance.

11. The method of claim 10, wherein the first wavelength is in a visible ray (VIS) region, and the second wavelength is in a short near infrared ray (SNIR) region.

12. The method of claim 10, wherein the third wavelength is a wavelength in a near-infrared ray (NIR) region, which is longer than a wavelength in a short near infrared ray (SNIR) region.

13. The method of claim 10, wherein the measuring of the first scattering coefficient and the measuring of the second scattering coefficient respectively comprise measuring the first scattering coefficient and the second scattering coefficient by using at least one of spatially resolved spectroscopy, time resolved spectroscopy, and frequency modulation spectroscopy.

14. The method of claim 10, wherein the obtaining of the third scattering coefficient comprises obtaining the third scattering coefficient from the first scattering coefficient and the second scattering coefficient by using monotonicity properties of the first scattering coefficient and the second scattering coefficient.

15. The method of claim 14, wherein the obtaining of the third scattering coefficient comprises deriving a monotonic function, representing the monotonicity properties of the first scattering coefficient and the second scattering coefficient, based on the first scattering coefficient and the second scattering coefficient, and obtaining the third scattering coefficient by using the derived monotonic function.

16. The method of claim 10, wherein the estimating of the concentration of the analyte comprises:
obtaining an absorption coefficient of the object at the third wavelength based on the obtained third scattering coefficient and the measured reflectance; and
estimating the concentration of the analyte by analyzing the obtained absorption coefficient.

17. The method of claim 16, wherein the obtaining of the absorption coefficient comprises:
obtaining an albedo of the object at the third wavelength based on the reflectance; and
obtaining the absorption coefficient based on the obtained albedo and the third scattering coefficient.

18. The method of claim 10, wherein the analyte is at least one of glucose, triglyceride, urea, uric acid, lactate, protein, cholesterol, and ethanol.

19. The apparatus of claim 1, further comprising the first optical sensor.

20. The apparatus of claim 19, further comprising the second optical sensor.
